(19) United States
(12) Patent Application Publication
(10) Pub. No.: US 2012/0083659 A1
(43) Pub. Date: Apr. 5, 2012

(54) Foam Surgical Retractor

(76) Inventor: Gerardo A. Caballero, Brookfield, WI (US)

(21) Appl. No.: 12/898,423

(22) Filed: Oct. 5, 2010

(51) Int. Cl. A61B 1/32

(52) U.S. Cl. 600/206

(57) ABSTRACT

There is a method and surgical retractor provided which includes a member having a first end and a second end. The member defines a concave inner side between the two ends. The member also defines a convex outer side between the two ends. The member includes a first side and a second side wherein the edge of the first side extends, between the two ends, beyond an edge of the second side. The surgical retractor may further comprise a V-shaped channel defined in the convex inner side. The member may be composed of a surgical foam, wherein the surgical foam includes a characteristic of restorative shape memory.

### BACKGROUND OF THE INVENTION

### Field of the Invention

The present invention relates to surgical retractors, and more particularly to a foam surgical retractor that does not need additional clamps or additional retractors to hold the foam surgical retractor in position in an abdominal cavity.

In the course of abdominal surgery, it typically is necessary to keep the surgical field clear of organs that are not part of the surgical procedure. For example, gauze laparotomy sponges (also referred to as lap sponges) and mechanical retractors are used in the abdominal cavity to keep the, for example, small bowel out of the way when the large bowel is the subject of the operation.

It is known to use cotton sponges which have been used without significant improvements, except for the attachment of radio-opaque markers for ease of x-ray identification. Frequently, multiple sponges are used to maintain visualization in the abdominal cavity. As a result, a “sponge count” at the end of the operation is necessary to insure that no lap sponges are left in the patient.

It is also known that self-retaining retractors are used to keep the abdominal wall open and the surgical field clear of organs that interfere with visualization of the organ to be operated on. Typically the systems include attachable blades and sponges and can be composed of mechanical blades and supporting arms and similar devices.

The apparatus of the present disclosure must also be of construction which is both durable and long lasting, and it should also require little or no maintenance to be provided by the user throughout its operating lifetime. In order to enhance the market appeal of the apparatus, it should also be of inexpensive construction to thereby afford it the broadest possible market. Finally, it is also an objective that all of the aforesaid advantages and objectives be achieved without incurring any substantial relative disadvantage.

### SUMMARY OF THE INVENTION

The disadvantages and limitations of the background art discussed above are overcome by the present disclosure.

A surgical retractor is provided which includes a member having a first end and a second end. The member defines a concave inner side between the two ends. The member also defines a convex outer side between the two ends. The member includes a first side and a second side wherein the edge of the first side extends, between the two ends, beyond an edge of the second side. The surgical retractor may further comprise a V-shaped channel defined in the convex inner side. The member may be composed of a surgical foam, wherein the surgical foam includes a characteristic of restorative shape memory.

There is further provided a method of maintaining retraction of an intestine during a surgical procedure. The method includes providing a member having a first and a second end, with the member defining a concave inner side between the two ends, and defining a convex outer side between the two ends. The member includes a first side and a second side, where an edge of the first side extends, between the two ends, beyond an edge of the second side. An incision is made in the abdominal wall of a patient and the member is compressed to a size sufficient for insertion in the incision. The compressed member is inserted into the incision and allowed to decompress once the member is inserted into the abdominal cavity. The member is positioned against a portion of the intestine with the concave inner side toward a surgical field within the patient. The member maintains the portion of intestine away from the surgical field by restorative shape memory of the member and serosal surface friction with the member. Compression of the member is facilitated by a V-shaped channel defined in the concave inner side of the member.

There is additionally provided a surgical retractor including a member having a first end and a second end with the member composed of a surgical foam. The surgical foam includes a characteristic of restorative shape memory and further defined a concave inner side between the two ends with the concave inner side defining a V-shaped channel. The member also defines a convex outer side between the two ends, with the member including a first side and a second side, wherein an edge of the first side extends, between the two ends, beyond an edge of the second side. The member may include an extension portion coupled to one end of the member.

The apparatus of the present disclosure is of a construction which is both durable and long lasting, and which will require little or no maintenance to be provided by the user throughout its operating lifetime. The apparatus of the present surgical retractor is also of inexpensive construction to enhance its market appeal and to thereby afford it the broadest possible market. Finally, all of the aforesaid advantages and objectives are achieved without incurring any substantial relative disadvantage.

### DESCRIPTION OF THE DRAWINGS

These and other advantages of the present disclosure are best understood with reference to the drawings, in which:

FIG. 1 is a schematic illustration of a patient with an exemplary embodiment of a foam surgical retractor disposed in the abdominal cavity of the patient.

FIG. 2A is a schematic cross-section illustration of the abdominal cavity of the patient illustrated in FIG. 1 along the line 2B-2B and illustrating a foam surgical retractor compressed for insertion into the abdominal cavity through an incision in the abdominal wall.

FIG. 2B is a schematic cross-section illustration of the abdominal cavity of the patient illustrated in FIG. 1 along the line 2B-2B and illustrating the foam surgical retractor illustrated in FIG. 1 retracting an intestine with the convex side of the foam surgical retractor and with a first and second side of the retractor in contact with the abdominal wall after decompression of the retractor.

FIG. 3 is a perspective view of an exemplary embodiment of a foam surgical retractor.

FIG. 4 is a partial sectional view of the foam surgical retractor illustrated in FIG. 3 along the line 4-4.

FIG. 5 is a plan view of the foam surgical retractor illustrated in FIG. 3 towards a V-shaped channel defined by a concave side of the retractor.

FIG. 6 is a plan view of the foam surgical retractor illustrated in FIG. 3 towards a second side of the retractor and illustrating an extension of an edge of a first side of the retractor extending beyond an edge of the second side of the retractor.

### DETAILED DESCRIPTION OF THE EXEMPLARY EMBODIMENTS

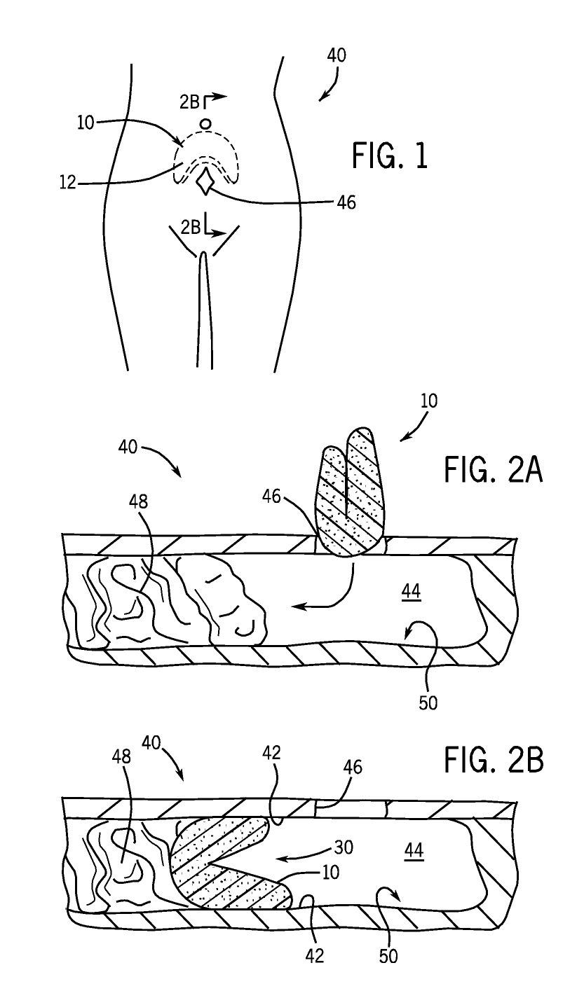

In the course of surgical procedures, for example colon resections, current clinical practices range from laparotomy to laparoscopic techniques. The trend in such colon resection surgeries tend to be more use of laparoscopic techniques resulting in shorter hospital stays, early return of bowel function and less expense. Further, the foam surgical retractor disclosed facilitates smaller incisions during surgical procedures. During such abdominal surgeries (See FIGS. 1-2B), a small incision 46 is made in the abdominal wall 42 of a patient 40 to expose the abdominal cavity 44 of the patient 40 so that the surgeon can operate on the intestine 48. It is necessary to keep a surgical field 50 within the abdominal cavity 44 clear of other organs and materials to allow the physician to operate in such confined space or through such small incisions 46.

In abdominal surgical procedures, lap-sponges of gauze-like material is often used to hold the organs, such as intestines, away from the operative area within the surgical field 50. Such gauze-like material is typically frequently replaced during an operation and also lacks the desired stiffness to hold the intestines in a particular place in all cases. It is known to use retractors of metal and rods to hold the lap sponges against organs to hold them away from the surgical field and in some cases to keep the incision 46 in the abdominal wall 42 of the patient 40 open and clear. Some surgical retractors composed of cushion-like members but require mechanical means to hold such retractors in place. Some retractors have a foam exterior but require or include a metal or stiff core structure internal to the cushion retractor to stiffen it for purposes of maintaining position and keeping organs not being operated on out of the surgical field.

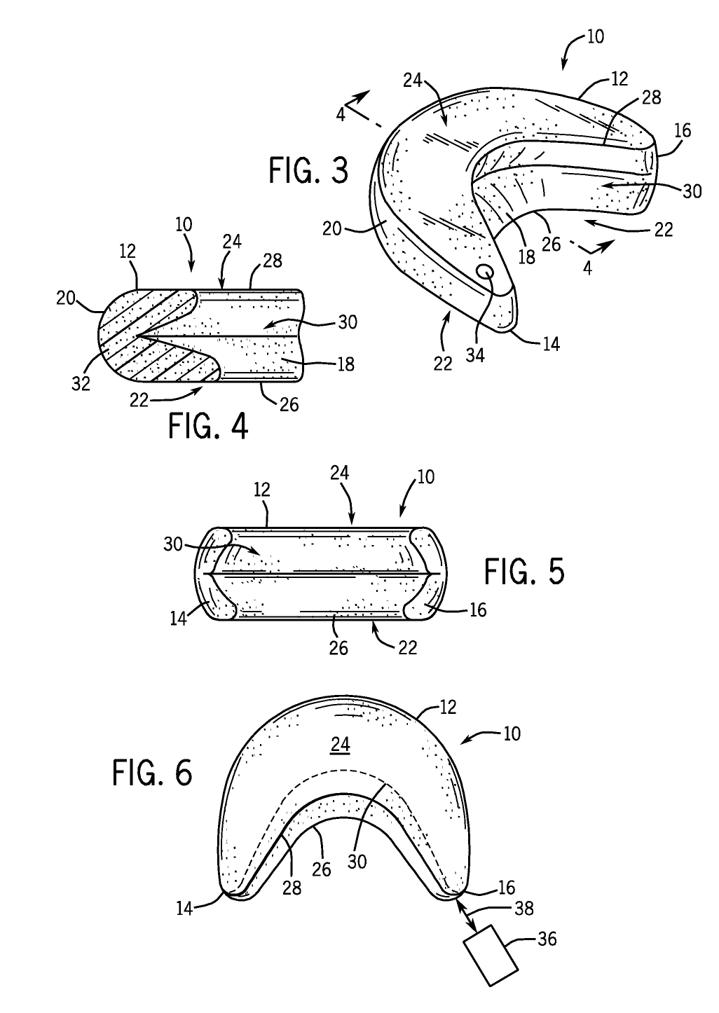

Referring to the Figures, FIG. 3 of the present disclosure provides a surgical retractor 10 than can be sized to meet different patient body sizes and is generally a “C” shape that tapers towards the periphery, defining a convex side 20. The deepest part of the surgical retractor 10 is located in a concave side 18 of the surgical retractor 10 providing maximum visualization within the surgical field 50 of the abdominal cavity 44. No additional retractors are needed to hold the surgical retractor 10 in place and the surgical retractor 10 does not include or require an interior core structure.

The surgical retractor 10 is a member 12 having a first end 14 and a second end 16. The member 12 defines a concave inner side 18 between the two ends (14, 16) and defines a convex outer side 20 between the two ends. The member 12 includes a first side 22 and a second side 24 wherein an edge 26 of the first side 22 extends, between the two ends, beyond an edge 28 of the second side 24. (See FIGS. 4 and 6)

The member 12 is configured that, in use, the first side 22 can be the bottom of the member 12 when the member 12 is in position within the abdominal cavity 44. (See FIGS. 4 and 2B) However, as determined by the surgeon during use, either the first side 22 or the second side 24 can be positioned as the top of the member 12 within the abdominal cavity 44.

The surgical retractor 10 further includes a V-shaped channel 30 defined in the concave side 18. (See FIGS. 3, 4, and 6) The V-shape channel facilitates the insertion of the member 12 into the incision 46 in the abdominal wall 42 of a patient 40 which results in the minimizing by compression of the overall size of the member 12 during the insertion process. (See FIG. 2A) Once the member 12 is fully inserted into the abdominal cavity 44 through the incision 46 the first side 22 and second side 24 of the member 12 decompresses because of a springing action of the surgical foam 32 from which the member 12 is composed. (See FIG. 2B) There is no rigid structure within the surgical foam 32 of the member 12.

The surgical foam 32 from which the member 12 is composed includes a characteristic of restorative shape memory thereby allowing the member 12 to resume its C-shape and the V-shape channel to open after insertion into the incision 46 by the surgeon. The surgical foam 32 can be of a material selected from a group consisting of polyurethane, polyvinylalcohol, biodegradable plastarch and synaptic foam.

In addition to the restorative shape memory of the surgical foam, once the member 12 is in position against an organ in the abdominal cavity 44, for example the large intestine 48, serosal surface friction with the member 12 of the surgical retractor 10 and the abdominal wall 42 within the abdominal cavity 44 maintains the position of the member 12 thereby keeping the intestine 48 out of the surgical field 50. (See FIG. 2B) As such, no additional mechanical clamping is required.

It should also be noted that because the size of the member 12 can be configured for the particular operation and patient, a single member 12 is all that may be required to maintain a clear surgical field 50 for a particular operation. An advantage of such circumstances is that only a single member 12 has to be accounted for the at the end of the surgical procedure. However, an identifier device 34, for example a radio-opaque marker or a radiofrequency transmitter chip can be coupled, for example embedding, to the member 12 to facilitate extraction of the member 12 at the end of the surgical procedure. (See FIG. 3)

In some instances, an extension portion 36 can be coupled to one end 14, 16 of the member 12 as determined by the surgeon. The extension portion 32 is composed of the same surgical foam 32 as the member 12 and is coupled to the member 12 by a fastener, for example a hook-a-loop fastener 38, such as for example Velcro®. (See FIG. 6) In another embodiment, the extension portion 36 can be integral with the member as a single unit as determined by a manufacturer of the surgical retractor 10 and/or the surgeon.

It should be understood that the extension portion 36 can be any suitable shape, for example a fish-tail or a wedge-shape, as determined by the surgeon. It should also be understood that the extension portion 36 can be modified by the surgeon to fit the situation by cutting the surgical foam of the extension portion 36 as needed. The extension portion 36 can also be coupled to the member 12 by the surgeon suturing the extension portion 36 to the member 12 at any suitable location on the member 12.

The surgical retractor 10 also defines a convex outer side 20 between the two ends of the member 12. It is the convex outer side 20 that is in direct contact with the organ being retracted and can be provided with a hydrophilic gel to decrease micro-trauma to the serosal surface of the abdominal wall 42.

Another advantage of the C-shaped member 12 is that the member 12 can be rotated, to position the concave inner side 18, to whichever quadrant within the abdominal cavity 44 that needs organ positioning and maintain a clear surgical field 50. The surgical foam 32 of the member 12 will also retain fluid which tends to keep an organ, for example a large intestine, hydrated during a surgical procedure.

During a surgical procedure on a patient, an incision 46 is made in the abdominal wall 44 of the patient 40. (See FIG. 1) A provided member 12 is compressed to a size sufficient for insertion into the incision 46. (See FIG. 2A) Because of the V-shape channel defined in the concave inner side 18 of the member 12, the surgeon compresses the member 12 by pressing on the first side 22 and the second side 24 of the member 12 immediately adjacent to the incision, and pushing the member 12 into the abdominal cavity 44. Consecutive compressions of the member 12 would proceed along the length of the member 12 until the entire member is inside the abdominal cavity 44. Other methods of compression and orientation of the member 12 can be employed by the surgeon to compress the member 12 prior to insertion into the incision 46.

Once the member 12 is inside the abdominal cavity 44 the surgeon would positions the member 12 against the intestine 48 at a location selected by the surgeon. The convex outer side 20 of the member 12 would maintain retraction of the intestine 48 with the surgical field 50 being within or bounded by the concave inner side 18 of the member 12. Once the member is fully inserted in the abdominal cavity 44 the restorative shape memory of the member 12 decompresses the surgical foam 32 as the first side 22 and second side 24 forming the V-shape channel 30 opens and serosal surface friction with the abdominal wall 42 and the member 12 maintains the position of the member 12.

If the surgeon determines that additional length of the member 12 is needed, an extension portion can be coupled to one or both ends 14, 16 of the member 12 with a hook-and-loop fastener 38.

At the end of the surgical procedure, the surgical retractor 10 is extracted from the abdominal cavity 44 through the incision 46. If there is any question that all or any of the surgical retractor 10 has not been fully extracted from the abdominal cavity 44, the identifier device 34 can be utilized to either confirm complete extraction or locate the retractor 10 in the abdominal cavity 44 of the patient 40.

It is contemplated that the surgical retractor 10 can be utilized in veterinary surgical procedures as well as in human surgical procedures.

For purposes of this disclosure, the term “coupled” means the joining of two components (electrical or mechanical) directly or indirectly to one another. Such joining may be stationary in nature or moveable in nature. Such joining may be achieved with the two components (electrical or mechanical) and any additional intermediate members being integrally formed as a single unitary body with one another or the two components and any additional member being attached to one another. Such adjoining may be permanent in nature or alternatively be removable or releasable in nature.

Although the foregoing description of the present foam surgical retractor has been shown and described with reference to particular embodiments and applications thereof, it has been presented for purposes of illustration and description and is not intended to be exhaustive or to limit the disclosure to the particular embodiments and applications disclosed. It will be apparent to those having ordinary skill in the art of abdominal surgery that a number of changes, modifications, variations, or alterations to the foam surgical retractor as described herein may be made, none of which depart from the spirit or scope of the present disclosure.

The particular embodiments and applications were chosen and described to provide the best illustration of the principles of the surgical retractor and its practical application to thereby enable one of ordinary skill in the art of abdominal surgery to utilize the retractor in various embodiments and with various modifications as are suited to the particular use contemplated. All such changes, modifications, variations, and alterations should therefore be seen as being within the scope of the present disclosure as determined by the appended claims when interpreted in accordance with the breadth to which they are fairly, legally, and equitably entitled.

What is claimed is:

1. A surgical retractor comprising:
a member having a first end and a second end, the member defining a concave inner side between the two ends, and defining a convex outer side between the two ends, the member including a first side and a second side, wherein an edge of the first side extends, between the two ends, beyond an edge of the second side.

2. The surgical retractor of claim 1, further comprising a V-shape channel defined in the concave inner side.

3. The surgical retractor of claim 1, wherein the member is composed of a surgical foam, wherein the surgical foam includes a characteristic of restorative shape memory.

4. The surgical retractor of claim 3, wherein the foam is one of a material selected from a group consisting of polyurethane, polyvinylalcohol, biodegradable plastarch foam, and silastic foam.

5. The surgical retractor of claim 1, including an identifier device coupled to the member

6. The surgical retractor of claim 1, further comprising an extension portion coupled to one end of the member.

7. The surgical retractor of claim 6, wherein the extension portion is one of integral with the member as a single unit and coupled to the member with a hook-and-loop fastener.

8. A method of maintaining retraction of an intestine during a surgical procedure comprising:
providing a member having a first end and a second end, the member defining a concave inner side between the two ends, and defining a convex outer side between the two ends, the member including a first side and a second side, wherein an edge of the first side extends, between the two ends, beyond an edge of the second side;
making an incision in an abdominal wall of a patient;
compressing the member to a size sufficient for insertion into the incision;
inserting the member into the incision;
allowing the member to decompress;
positioning the member against a portion of the intestine with the concave inner side toward a surgical field within the patient, wherein the member maintains the portion of intestine away from the surgical field by restorative shape memory of the member and serosal surface friction with the member.

9. The method of maintaining retraction of an intestine of claim 8, further comprising facilitating the compression of the member with a V-shape channel defined in the concave inner side.

10. The method of maintaining retraction of an intestine of claim 8, wherein the member is composed of a surgical foam, with the surgical foam including a characteristic of restorative shape memory.

11. The method of maintaining retraction of an intestine of claim 10, wherein the surgical foam is one of a material selected from a group consisting of polyurethane, polyvinylalcohol, biodegradable plastarch foam, and silastic foam.

12. The method of maintaining retraction of an intestine of claim 8, including an identifier device coupled to the member.

13. The method of maintaining retraction of an intestine of claim 8, further comprising providing an extension portion coupled to one end of the member.

14. The method of maintaining retraction of an intestine of claim 13, wherein the extension portion is integral with the member as a single unit.

15. The method of maintaining retraction of an intestine of claim 13, including coupling the extension portion to the member with a hook-and-loop fastener.

16. A surgical retractor comprising:
a member having a first end and a second end with the member composed of a surgical foam, wherein the surgical foam includes a characteristic of restorative shape memory, the member further defining a concave inner side between the two ends with the concave inner side defining a V-shape channel, and the member defining a convex outer side between the two ends, the member including a first side and a second side, wherein an edge of the first side extends, between the two ends, beyond an edge of the second side.

17. The surgical retractor of claim 16, wherein the surgical foam is one of a material selected from a group consisting of polyurethane, polyvinylalcohol, biodegradable plastarch foam, and silastic foam.

18. The surgical retractor of claim 16, including an identifier device coupled to the member.

19. The surgical retractor of claim 16, further comprising an extension portion coupled to one end of the member.

20. The surgical retractor of claim 19, wherein the extension portion is one of integral with the member as a single unit and coupled to the member with a hook-and-loop fastener.
